(19) United States
(12) Patent Application Publication
(10) Pub. No.: US 2006/0233882 A1
(43) Pub. Date: Oct. 19, 2006

(54) Osmotic dosage form

(76) Inventor: Harry S. Sowden, Glenside, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/107,763

(22) Filed: Apr. 15, 2005

(51) Int. Cl. A61K 9/24

(52) U.S. Cl. 424/473

(57) ABSTRACT

The present invention is directed to a modified release dosage form for delivering at least one pharmaceutically active ingredient. The dosage form has a first immediate release core for an active ingredient and an osmotic core or osmotic chamber containing at least one pharmaceutically active ingredient that can be the same or a different active ingredient contained in the first immediate release core. A shell having one or more portions surrounds the first immediate release core and osmotic core/chamber. The osmotic chamber includes a barrier layer that is substantially impermeable to the pharmaceutically active ingredient contained therein.

### FIELD OF THE INVENTION

This invention relates to dosage forms providing modified release of one or more active ingredients contained therein via an osmotic delivery system.

### BACKGROUND OF THE INVENTION

Modified release pharmaceutical dosage forms have long been used to optimize drug delivery and enhance patient compliance, especially by reducing the number of doses of medicine the patient must take in a day. In some instances, it is also desirable for a dosage form to deliver more than one drug at different rates or times. Modified release dosage forms should ideally be adaptable so that release rates and profiles can be matched to physiological and chronotherapeutic requirements. Because the onset and duration of the therapeutic efficacy of drugs vary widely, as do their absorption, distribution, metabolism, and elimination, it is often desirable to modify the release of different drugs in different ways, or to have a first dose of drug (active ingredient) immediately released from the dosage form, while a second dose of the same or a different drug is released in a modified, e.g. delayed, pulsatile, repeat action, controlled, sustained, prolonged, extended, or retarded manner.

Well-known mechanisms by which a dosage form (or drug delivery system) can deliver drug at a controlled rate (e.g. sustained, prolonged, extended or retarded release) include diffusion, erosion, and osmosis. It is often practical to design dosage forms that use a combination of the above mechanisms to achieve a particularly desirable release profile for a particular active ingredient.

An important objective of modified release dosage forms is to provide a desired blood concentration versus time (pharmacokinetic, or PK) profile for the drug. Fundamentally, the PK profile for a drug is governed by the rate of absorption of the drug into the blood, and the rate of elimination of the drug from the blood. To be absorbed into the blood (circulatory system), the drug must first be dissolved in the gastrointestinal fluids. For those relatively rapidly absorbed drugs whose dissolution in gastro-intestinal fluids is the rate-limiting step in drug absorption, controlling the rate of dissolution (i.e. drug release from the dosage form) allows the formulator to control the rate of drug absorption into the circulatory system of a patient. The type of PK profile, and correspondingly, the type of dissolution or release profile desired, depends on, among other factors, the particular active ingredient and physiological condition being treated.

One particularly desirable PK profile is achieved by a dosage form that delivers a delayed release dissolution profile, in which the release of one or more doses of drug from the dosage form is delayed for a pre-determined time after contacting of the dosage form by a liquid medium, such as for example, after ingestion by the patient. The delay period (“lag time”) can be followed either by prompt release of the active ingredient (“delayed burst”), or by sustained (prolonged, extended, or retarded) release of the active ingredient (“delayed then sustained”). U.S. Pat. No. 5,464,633, for example, discloses delayed-release dosage forms in which an external coating layer was applied by a compression coating process. The coating level ranged from 105 percent to 140 percent of the weight of the core in order to yield product with the desired time delayed profile.

One particularly desirable type of delayed release PK profile is obtained from a “pulsatile” release profile, in which for example, a first dose of a first drug is delivered, followed by a delay period (“lag time”) during which there is substantially no release of the first drug from the dosage form, followed by either prompt or sustained release of a subsequent dose of the same drug. In one particularly desirable type of pulsatile drug delivery system, the first dose is released essentially immediately upon contacting of the dosage form with a liquid medium. In another particularly desirable type of pulsatile drug delivery system, the delay period corresponds approximately to the time during which a therapeutic concentration of the first dose is maintained in the blood. Pulsatile delivery systems are particularly useful for applications where a continuous release of drug is not ideal. Examples of this are drugs exhibiting first pass metabolism by the liver, drugs that induce biological tolerance, i.e. the therapeutic effect decreases with continuous presence of the drug at the site of action, and drugs whose efficacy is influenced by circadian rhythms of body functions or diseases. One typical pulsatile dosage form design contains the first dose of drug in an exterior coating, or shell, while subsequent doses of drug are contained in underlying layers of subcoatings, or a central core. PCT Publication No. WO 99/62496, for example, discloses a dosage form comprising an immediate-release dose of drug contained within an overcoat applied onto the surface of the semi-permeable membrane of an osmotic dosage form. U.S. Pat. Nos. 4,857,330 and 4,801,461, disclose dosage forms comprising an exterior drug coat that surrounds a semi-permeable wall, which in turn surrounds an internal compartment containing a second dose of drug, and comprises exit means for connecting the interior of the dosage form with the exterior environment of use. These dosage forms are designed to release drug immediately from the exterior coating, followed by a relatively short delay period, followed by a sustained release of drug from the internal compartment.

U.S. Pat. No. 4,576,604, for example, discloses an osmotic device (dosage form) comprising a drug compartment surrounded by a wall (coating) having a passageway therein. The wall may comprise an immediate release dose of drug, and the inner drug compartment may comprise a sustained release dose of drug. U.S. Pat. No. 4,449,983 discloses another osmotic device comprising two separately housed drugs that are separately dispensed from the device. The device comprises two compartments, one for each drug, separated by a partition. Each compartment has an orifice for communicating with the exterior of the device. U.S. Pat. No. 5,738,874, discloses a 3-layer pharmaceutical compressed tablet capable of liberating one or more drugs at different release rates, in which an immediate release dose of active may be contained in a compressed coating layer, and in one embodiment, the outer compressed coating layer may function via an erosion mechanism to delay release of a second dose of active ingredient contained in the core. Systems such as these are limited by the amount of drug, which may be incorporated into the exterior coating, or shell, which is in turn limited by the achievable thickness of the exterior coating or shell.

Another design for a pulsatile delivery system is exemplified in U.S. Pat. No. 4,865,849, which describes a tablet able to release active substances at successive times, comprising a first layer containing a portion of the active substance, a water soluble or water gellable barrier layer which is interposed between the first layer and a third layer containing the remaining portion of active substance, and the barrier layer and third layer are housed in an insoluble, impermeable casing. The casing can be applied by various methods such as spraying, compression, or immersion, or the tablet parts can be inserted into a pre-formed casing. Multilayer compressed tablets in stacked layer configurations necessarily require an impermeable partial coating (casing) in order to provide a pulsatile release profile. These systems suffer from the complexity and high cost of assembling multiple, separate compartments comprising multiple, different compositions.

Dosage forms have been previously designed with multiple cores housed in a single shell for the purpose of allowing flexibility in a dosing regimen. PCT Publication No. WO 00/18447, for example, describes a multiplex drug delivery system suitable for oral administration containing at least two distinct drug dosage packages, which exhibit equivalent dissolution profiles for an active agent when compared to one another and when compared to that of the entire multiplex drug delivery unit, and substantially enveloped by a scored compressed coating that allows the separation of the multiplex drug delivery system into individual drug dosage packages. In this example, two immediate-release compartments are enveloped by a scored extended-release compartment. Active ingredient may be contained in only the extended release compartment, or additionally in the two immediate release compartments. The multiplex drug delivery systems of this example are prepared by press coating the extended-release compartment to substantially envelop the immediate release compartments.

Published U.S. patent application 2003/0235616 describes a modified release dosage form comprising at least one active ingredient and at least two cores surrounded by a shell. The shell comprises at least one opening.

Published U.S. patent application 2003/0232082 describes a modified release dosage form comprising at least one active ingredient, a core having an outer surface, and a shell that resides upon at least a portion of the core outer surface and a shell that is semi-permeable such that the liquid medium diffuses through the shell to the core due to osmosis.

Osmotic dosage forms for delivering a drug to a fluid environment of use are known to the drug dispensing art. For example, U.S. Pat. No. 3,845,770 and in U.S. Pat. No. 3,916,899, an osmotic dosage form is disclosed comprising a semipermeable wall that surrounds a compartment comprising a drug. The wall is permeable to the passage of fluid and there is a passageway through the wall for delivering the drug from the dosage form. The dosage forms of these patents, release the drug by fluid being imbibed through the wall into the compartment at a rate determined by the permeability of the wall and the osmotic pressure gradient across the wall to produce a solution of drug that is dispensed through the passageway from the dosage form. These dosage forms are extraordinarily effective for delivering a drug that exhibits an osmotic pressure gradient across the wall against the fluid. The dosage forms are effective also for delivering a drug mixed with an osmotically effective solute that is soluble in the fluid and exhibits an osmotic pressure gradient across the wall against an aqueous fluid.

An improvement in osmotic dosage forms was presented to the medical and pharmaceutical dispensing art in U.S. Pat. Nos. 4,111,202; 4,111,203; and 4,203,439. In these patents, the delivery kinetics of the dosage form was enhanced for delivering a drug with varying degrees of solubility in an aqueous fluid. The kinetics are improved by manufacturing the dosage form with a drug compartment and an osmotic compartment separated by a film. These dosage forms deliver the drug by fluid being imbibed through the wall into the osmotic compartment producing a solution that causes the film to move and act as a driving force. The driving force pushes the drug through a small passageway from the dosage form.

Further advances in osmotic dosage forms were described in U.S. Pat. No. 4,327,725 and in U.S. Pat. No. 4,612,008. The osmotic dosage forms in these patents comprise a semipermeable wall that surrounds a compartment. The compartment contains a drug formulation and an expandable hydrogel. In operation, fluid is imbibed into the compartment where it contacts the drug formulation; thereby forming a dispensable formulation that is pushed by the expanding hydrogel from the dosage form.

U.S. Pat. No. 4,627,850 describes an osmotic capsule comprising a wall encapsulating and containing a drug formulation. The drug formulation is delivered through a very small orifice manufactured without any expressed ratio to the dimensions of the osmotic capsule.

Improved dosage forms for providing modified release of active ingredient are described herein, particularly as osmotic drug delivery devices.

### SUMMARY OF THE INVENTION

The present invention is directed, in one aspect, to a dosage form having a first immediate release core that contains at least one pharmaceutically active ingredient, an osmotic core that contains at least one pharmaceutically active ingredient that is the same or different from the pharmaceutically active ingredient provided in the first core, and a unitary shell that conforms and coats at least a substantial portion of both the first immediate release core and the osmotic core. The shell material is substantially impermeable to the pharmaceutically active ingredient in the osmotic core. Further, at least one passageway can be provided through the shell to the immediate release core with sufficient size for immediate release of the active contained therein and at least one passageway can be provided through the unitary shell to the osmotic chamber. The passageways can be provided with a fill material that is compositionally different from the shell. The first immediate release core can be a multi-layer tablet. The pore volume of the shell as applied to the dosage form is preferably less than 0.2 cc/g for pores having a diameter between about 0.5 to about 5 microns.

The present invention is directed, in another aspect, to a dosage form having a first immediate release core that contains at least one pharmaceutically active ingredient, an osmotic core that contains at least one pharmaceutically active ingredient that is the same or different from the pharmaceutically active ingredient provided in the first immediate release core, and a shell having distinct, compositionally different portions that as a whole conform and coat at least a substantial portion of both the first immediate release core and the osmotic core. The shell has a major portion consisting essentially of material that is substantially impermeable to the pharmaceutically active ingredient in the osmotic chamber and a second portion of the shell that is in contact with the immediate release core that consists essentially of immediate release material. At least one passageway can be provided through the shell to the osmotic chamber. The first immediate release core can be a compressed solid tablet. The first immediate release core can be a multi-layer tablet. All portions of the shell can be substantially free of pores having a diameter of 0.5 to 5.0 microns. The pore volume of all portions of the shell as applied to the dosage form is preferably less than 0.2 cc/g for pores having a diameter between about 0.5 to about 5 microns.

The present invention relates, in another aspect, to a dosage form having a first immediate release core that contains at least one pharmaceutically active ingredient, an osmotic core that contains at least one pharmaceutically active ingredient that is the same or different from the pharmaceutically active ingredient provided in the first immediate release core, and a unitary shell that conforms and coats at least a substantial portion of the first immediate release core and the osmotic core. A portion of the shell provided over the immediate release core is sufficiently thin that it ruptures upon swelling of the immediate release core to release the active contained therein. At least one passageway can be provided through the shell to the osmotic chamber. The first immediate release core can be a compressed solid tablet. The shell is preferably substantially free of pores having a diameter of 0.5 to 5.0 microns. Alternatively, the pore volume of the shell as applied to the dosage form is less than 0.2 cc/g for pores having a diameter between about 0.5 to about 5 microns.

The present invention further relates to a dosage form having a first immediate release core that contains a pharmaceutically active ingredient, an osmotic chamber that contains one pharmaceutically active ingredient that is the same or different from the pharmaceutically active ingredient provided in the first core, and a shell that consists essentially of an immediate release material and coats at least a substantial portion of the first immediate release core and the osmotic chamber. The osmotic chamber includes a barrier layer that is substantially impermeable to the active ingredient contained therein. A passageway can be provided through the barrier layer. The first immediate release core can be a compressed solid tablet. The shell as applied to the dosage form is preferably substantially free of pores having a diameter of 0.5 to 5.0 microns. Alternatively, the pore volume of shell as applied to the dosage form is less than 0.2 cc/g for pores having a diameter between about 0.5 to about 5 microns.

The present invention further relates to a dosage form having a first core that contains a pharmaceutically active ingredient, an osmotic chamber that contains one pharmaceutically active ingredient that is the same or different from the pharmaceutically active ingredient provided in the first core, and a shell having distinct, compositionally different portions that coat a substantial portion of the first core and osmotic chamber. The first shell portion that is in contact with the first core provides an immediate release of the pharmaceutically active ingredient in the first core and the second shell portion in contact with the osmotic chamber produces a modified release profile. The first immediate release core can be a compressed solid tablet or a multi-layer tablet. All portions of the shell are preferably substantially free of pores having a diameter of 0.5 to 5.0 microns. The pore volume of all portions of the shell as applied to the dosage form are preferably less than 0.2 cc/g for pores having a diameter between about 0.5 to about 5 microns.

The present invention further relates to a method for preparing a dosage form by providing a first immediate release core containing at least one pharmaceutically active ingredient and an osmotic core or an osmotic chamber containing at least one pharmaceutically active ingredient that is the same or different from the pharmaceutically active ingredient provided in the first core to shell-forming module, and providing a shell that conforms and coats at least a substantial portion of both the first immediate release core and the osmotic core or osmotic chamber.

The present invention also relates to a method for preparing a dosage form by providing a first immediate release core containing at least one pharmaceutically active ingredient and an osmotic core or an osmotic chamber containing at least one pharmaceutically active ingredient that is the same or different from the pharmaceutically active ingredient provided in the first core to a shell-forming module, and providing a shell having distinct portions and that conforms and coats at least a substantial portion of both the first immediate release core and the osmotic core or osmotic chamber.

### BRIEF DESCRIPTION OF THE DRAWINGS

FIG. 1 is a side view of an osmotic chamber.

FIGS. 2 and 3 illustrate a sectioned view of an osmotic chamber.

FIG. 4 is an alternative embodiment of the present invention.

FIG. 5 is an alternative embodiment of the present invention.

FIG. 6 is an alternative embodiment of the present invention.

### DETAILED DESCRIPTION OF THE INVENTION

As used herein, the term “dosage form” applies to any solid object or semi-solid designed to contain a specific pre-determined amount (dose) of a certain ingredient, for example an active ingredient as defined below. Suitable dosage forms may be pharmaceutical drug delivery systems, including those for oral administration, buccal administration, rectal administration, topical or mucosal delivery, or subcutaneous implants, or other implanted drug delivery systems; or compositions for delivering minerals, vitamins and other nutraceuticals, oral care agents, flavorants, and the like. Preferably the dosage forms of the present invention are considered to be solid, however they may contain liquid or semi-solid components. In a particularly preferred embodiment, the dosage form is an orally administered system for delivering a pharmaceutical active ingredient to the gastro-intestinal tract of a human.

In one embodiment, the shell is understood to be a material that is malleable, flowable and conformable such that a shell material, when applied, will have a surface that conforms to the shape of the element (whether a core or another underlying shell) over which it is applied. With respect to this embodiment, the core, in contrast, has a fixed, generally unitary shape that does not change significantly when introduced or provided in the dosage form. A unitary shell is understood to mean a continuous layer of materials that has been provided over one or more components of the dosage form. It is possible for a unitary shell to be provided on the dosage form in multiple, separate steps provided the subsequent additions utilize substantially the same compositional materials.

The present invention is directed to a dosage form for delivering at least one active ingredient to a subject having a first immediate release core for a pharmaceutically active ingredient, an osmotic chamber comprising at least one core for a pharmaceutically active ingredient that can be the same or different from the immediate release core and an osmagent, and a shell that surrounds the first immediate release core and the osmotic chamber, wherein the osmotic chamber is surrounded, with the exception of an egress for controlled delivery of the pharmaceutically active ingredient, by an barrier layer that is impermeable to the pharmaceutically active ingredient contained therein.

In one embodiment, the first immediate release core is completely surrounded by or embedded in the shell material that has immediate release properties. In an alternative embodiment, the first immediate release core and the osmotic chamber are surrounded or embedded in a shell that provided a modified release profile. In a still further embodiment, the first immediate release core and the osmotic chamber are surrounded or embedded in a shell that provided a modified release profile while openings are provided through the shell in the vicinity of the first immediate release core. Still further, the first immediate release core and the osmotic chamber are surrounded or embedded in a shell having distinct portions such that the shell in the vicinity of the first immediate release core has immediate release properties, while the shell provided over at least the egress, preferably over any exposed surface area of the osmotic chamber has a modified release profile.

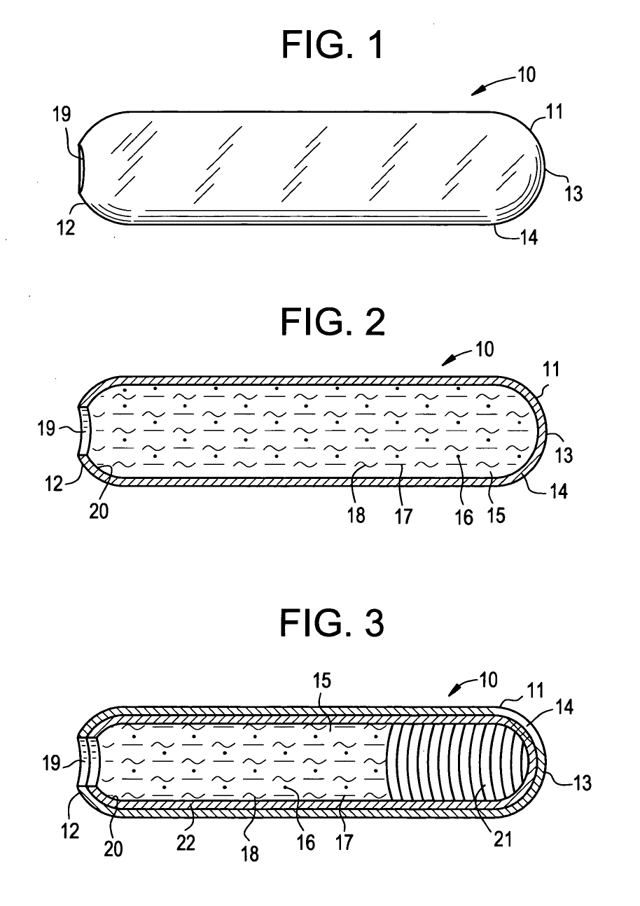

FIGS. 1 through 3 are illustrative of an osmotic chamber that can be manufactured as part of the present invention. In accordance with the practice of this invention, osmotic chamber 10 is manufactured with a semipermeable wall 14 that does not adversely affect drug 16, the components comprising osmotic chamber 10, and an animal, including a human patient host. Semipermeable wall 14 is permeable to the passage of external fluid such as water and biological fluids, and it is substantially impermeable to the passage of drug 16.

Compartment 15 comprises an effective amount of a pharmaceutically active ingredient or drug represented by dots. Drug 16 in one embodiment, is soluble to very soluble in an external fluid imbibed through semipermeable wall 14 into compartment 15, and drug 16 exhibits an osmotic pressure gradient across wall 14. Drug 16, in another embodiment exhibits a limited solubility in fluid imbibed into compartment 15, and it exhibits a limited osmotic pressure gradient across wall 14. In this latter embodiment, drug 16 optionally mixed with an osmagent 17, presented as dashes, which osmagent 17 is soluble in the external fluid and it exhibits an osmotic pressure gradient across wall 14 for aiding in dispensing drug 16 from osmotic chamber 10. Drug 16 can be present in compartment 15 with an optional member selected from the group consisting of a binder, dispersant, wetting agent, suspending agent, lubricant and dye, represented by wavy line 18. Representative of the members including suspending agents such as colloidal magnesium, silicon dioxide, and calcium silicate; binders like polyvinyl pyrrolidone, lubricants like magnesium stearate, and wetting agents such as fatty amines and fatty quaternary ammonium salts. A dye can be present in the compartment 15 for aiding in identifying a drug 16 present in osmotic chamber 10.

Passageway 19 is a wide-passageway that is generally cylindrical. In the preferred manufacture, passageway 19 is positioned at the tip of the convex surface of the drug surface 20 of wall 12. The passageway area optionally can be as large as the cylindrical cross-section area of the chamber. Caplet passageway 19, in another manufacture comprises a multiplicity of orifices in convex surface 20, with the total area of the multiplicity of caplet passageways 19 less than the cross-sectional area of the oblong osmotic caplet 10 at its widest area.

FIG. 3 depicts osmotic chamber 10 comprising a body 11 having a lead end 12, rear end 13, wall 14 and compartment 15. Osmotic core or alternatively compartment 15 comprises drug 16 and an osmopolymer 21, or an expandable driving member, identified by curve lines. Osmopolymer 21 is in contact with the drug 16 composition. The drug 16 composition layer and the osmopolymer 21 layer operate in union for delivering the maximum dose of drug 16 through opened passageway 19. Compartment 15 optionally comprises a member 18 selected from the group consisting of a binder, dispersant, wetting agent, suspending agent, lubricant, and dye. Osmotic chamber 10 comprises additionally an internal wall 22 that faces compartment 15 and is in layered arrangement with the internal surface of wall 14. Internal wall 22 is non-toxic and it does not adversely affect drug 16 and other members 18 present in compartment 15. Internal wall 22 is permeable to the passageway of aqueous and biological fluids, and it comprises a hydrophilic polymeric composition that swells in the presence of fluid imbibed into compartment 15.

The selectively semipermeable wall compositions are non-erodible, nontoxic, and they are insoluble in fluids. Typical materials for forming wall 14 in one embodiment are cellulose esters, cellulose ethers and cellulose ester-ethers. These cellulosic polymers have a degree of substitution, D.S., on the anhydroglucose unit from greater than 0 up to 3 inclusive. By degree of substitution is meant the average number of hydroxyl groups originally present on the anhydroglucose unit comprising the cellulose polymer that are replaced by a substituting group. Representative materials include a member selected from the group consisting of cellulose acylate, cellulose diacrylate, cellulose triacrylate, cellulose acetate, cellulose diacetate, cellulose triacetate, mono, di and tricellulose alkanylates, mono, di and tricellulose aroylates, and the like. Exemplary polymers include cellulose acetate having a D.S. up to 1 and acetyl content up to 21%; cellulose acetate having an acetyl content of 32 to 39.8%: cellulose diacetate having a D.S. of 1 of 2 and an acetyl content of 21 to 35%; cellulose triacetate having a D.S. of 2 to 3 and an acetyl content of to 44.8%; and the like. More specific cellulosic polymers include cellulose propionate having a D.S. of 1.8 and a propionyl content of 39.2 to 45% and a hydroxyl content of 2.8 to 5.4%; cellulose acetate-butyrate having a D.S. of 1.8, and acetyl content of 13 to 15% and a butyryl content of 34 to 39%; cellulose acetate butyrate having an acetyl content of 2 to 29%, a butyryl content of 17 to 53% and a hydroxyl content of 0.5 to 4.7%; cellulose triacylates having a D.S. of 2.9 to 3 such as cellulose trivalerate, cellulose trilaurate, cellulose tripalmitate, cellulose trisuccinate, and cellulose trioclanoate; cellulose diacylates having a D.S. of 2.2 to 2.6 such as cellulose disuccinate, cellulose dipalmitate, cellulose dioclanoate, cellulose dipentale, and the like.

Additional semipermeable polymers for wall 12 include acetaldehyde dimethyl acetate, cellulose acetate ethyl carbamate, cellulose acetate phthalate for use in environments having a low pH, cellulose acetate methyl carbamate, cellulose acetate dimethyl aminoacetate, semipermeable polyamides, semipermeable polyurethanes, semipermeable sulfonated polystyrenes, cross-linked selectively semipermeable polymers formed by the co-precipitation of a polyanion and a polycation as disclosed in U.S. Pat. Nos. 3,173,876; 3,276,586; 3,541,005; 3,541,006; and 3,546,142; semipermeable polymers as disclosed by Loeb and Sourirajan in U.S. Pat. No. 3,133,132; lightly cross-linked polystyrene derivatives; cross-linked poly(sodium styrene sulfonate), cross-linked poly(vinylbenzyltrimethyl ammonium chloride), semipermeable polymers exhibiting a fluid permeability of 10.sup.-5 to 10.sup.-1 (cc.mil/cm²hr.atm) expressed as per atmosphere of hydrostatic or osmotic pressure difference across the semipermeable wall. The polymers are known to the art in U.S. Pat. Nos. 3,845,770; 3,916,899; and 4,160,020; and in Handbook of Common Polymers by Scott, J. R. and Roff W. J., 1971, published by CRC Press, Cleveland, Ohio.

Inner wall 22, in contact with external wall 14 comprises in a presently preferred manufacturer gelatin. The gelatin used for the present purpose comprises a viscosity of 10 to 40 millipoises and a bloom strength up to 150 grams; and gelatin comprising a bloom value of 160 to 250. The inner wall 22 can comprises 100 weight percent gelatin, or in another manufacturer inner wall 22 can comprises 60 wt % to 99 wt % gelatin, and 1 wt % to 40 wt % of a polysaccharide selected from the group consisting of agar, acacia, karaya, tragacanth, algin and guar.

Osmagent 17 that can be used for the purpose of this invention comprises inorganic and organic, preferably a solid compound that exhibits an osmotic pressure gradient across an external fluid across semipermeable wall 14. The osmagents are known also as osmotically effective compounds, osmotic solutes, and osmotic fluid imbibing agents. The osmagents are used by mixing the osmagent with drug 16 that has a limited solubility in the external fluid to provide an osmotic solid composition, that after fluid is imbibed into caplet, an osmotic drug solution that is hydrodynamically and osmotically delivered from osmotic chamber 10.

The phrase limited solubility as used herein means the drug has a solubility of less than 1% by weight in an aqueous fluid present in the environment. The osmagents are used by homogenously or heterogeneously mixing the osmagent with the drug and then changing the blend into a caplet or pressing the blend into a shape corresponding to the shape of osmotic chamber 10, which is then surrounded with semipermeable wall 14. The osmagent attracts fluid into caplet 10 producing a solution which is delivered from the caplet concomitantly transporting undissolved and dissolved drug to the exterior of caplet 10. Osmagent 17 used for the purpose of this invention comprises a member selected from the group consisting of a salt, oxide, carbohydrate, acid, ester, magnesium sulfate, magnesium chloride, sodium chloride, potassium chloride, lithium chloride, potassium sulfate, sodium sulfate, lithium sulfate, lithium phosphate, sodium phosphate, potassium phosphate, potassium acid phosphate, calcium lactate, mannitol, urea, inositol, magnesium succinate, tartaric acid, carbohydrates such as raffinose, sucrose, glucose, lactose monohydrate, and a blend of fructose glucose.

The osmopolymer 17, operable for pushing drug 16 composition from caplet 10 comprises a hydrophilic polymer. Hydrophilic polymers are known also as osmopolymers. The osmopolymers interact with water and aqueous biological fluids and swell or expand to an equilibrium state. The osmopolymers exhibit the ability to swell in water and retain a significant portion of the imbibed water within the polymer structure. The osmopolymers swell or expand to a very high degree, usually exhibiting a 2 to 60-fold volume increase. The osmopolymers can be noncross-linked or cross-linked. The swellable, hydrophilic polymers are, in one presently preferred embodiment, lightly cross-linked, such as cross-links being formed by covalent or ionic bonds. The osmopolymers can be of plant, animal or synthetic origin. Hydrophilic polymers suitable for the present purpose include poly(hydroxyalkylmethacrylate) having a molecular weight of from 30,000 to 5,000,000; poly(vinylpyrrolidone) having molecular weight of from 10,000 to 360,000; anionic and cationic hydrogels; polyelectrolyte complexes, poly(vinyl alcohol) having a low acetate residual, cross-linked with flyoxal, formaldehyde, or glutaraldehyde and having a degree of polymerization from 200 to 30,000; a mixture of methyl cellulose, cross-linked agar and carboxymethyl cellulose; a water insoluble, water swellable copolymer reduced by forming a dispersion of finely divided copolymer of maleic anhydride with styrene, ethylene, propylene, butylene or isobutylene cross-linked with from 0.00001 to about 0.5 moles of polyunsaturated cross-linking agent per mole of maleic anhydride in the copolymer; water swellable polymers of N-vinyl lactams, and the like.

Other osmopolymers include hydrogel polymers such as Carbopol® acidic carboxy polymers generally having a molecular weight of 450,000 to 4,000,000; the sodium salt of Carbopol® acidic carboxy polymers and other metal salts; Cyanamer® polyacrylamides; cross-linked water swellable indenemaleic anhydride polymers; Goodrite® polyacrylic acid having, but not limited to, a molecular weight of 80,000 to 200,000, and the sodium and other metal salts; Polyox® polyethylene oxide polymers having a molecular weight of 100,000 to 7,500,000; starch graft copolymers; Aqua-Keeps® acrylate polymers; diester cross-linked polyglucan, and the like. Representative polymers that form hydrogels are known to the prior art in U.S. Pat. No. 3,865,108 issued to Hartop; U.S. Pat. No. 4,002,173 issued to Manning; U.S. Pat. No. 4,207,893 issued to Michaels, in U.S. Pat. No. 4,576,604 issued to Guittard et al.; in U.S. Pat. No. 4,855,141 issued to Eckenhoff et al.; and in Handbook of Common Polymers, by Scott and Roff, published by the Chemical Rubber, CRC Press, Cleveland, Ohio. Osmotic core 15 can be prepared by compression as a multilayered tablet in known fashion. An osmotic core, in contrast to the osmotic chamber, does not include one or more barrier layers.

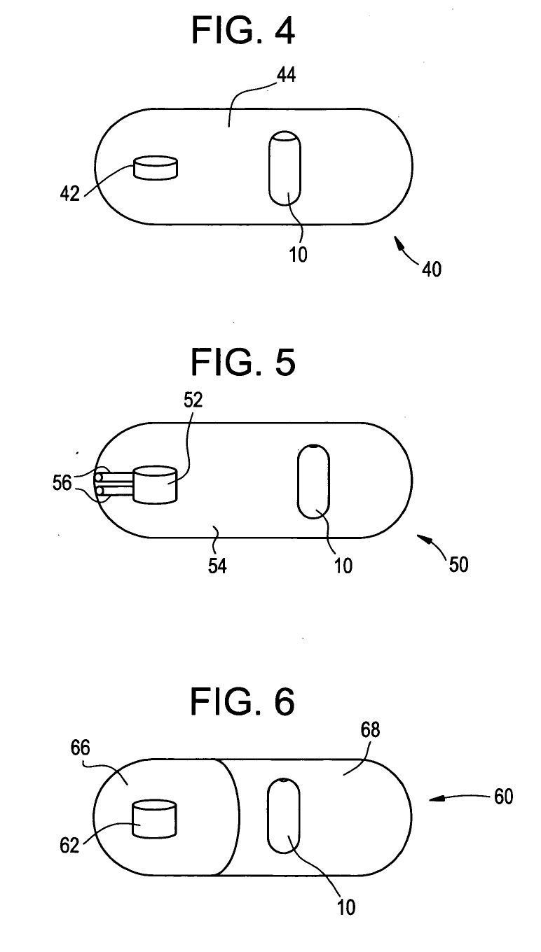

An exemplary dosage form 40 is shown in FIG. 4. Dosage form 40 comprises a first immediate release core 42 for a pharmaceutically active ingredient, osmotic chamber 10 described above, and a shell 44 that surrounds first immediate release core 42 and osmotic chamber 10. One advantage of the present invention is the ability to provide a large immediate release of pharmaceutical active from the immediate release core relative to the commercially reasonable dosing levels that can be achieved in a coating layer. Preferred dosing amounts for exemplary pharmaceutical actives are acetaminophen, at least 200 mg, preferably at least about 325 mg, alternatively about 500 mg; ibuprofen at least about 100 mg, preferably about 200 mg; aspirin at least about 100 mg, preferably about 325 mg; guafenisen at least about 100 mg, preferably about 200 mg; and calcium carbonate at least about 500 mg, preferably about 800 mg. One embodiment of the present invention provides an immediate release core containing at least about 100 mg of pharmaceutically active ingredient, alternatively at least about 200 mg, still further at least about 325, and still further at least about 500 mg.

In one embodiment, first immediate release core 42 is completely surrounded by or embedded in shell 44 that has immediate release properties. In an alternative embodiment, first immediate release core 42 and osmotic chamber 10 are surrounded by or embedded in shell 44 that provides a modified release profile. In a still further embodiment shown in FIG. 5, dosage form 50 comprises first immediate release core 52 and osmotic chamber 10, which are surrounded or embedded in shell 54 that provides a modified release profile and wherein at least one opening 56 is provided through the shell in the vicinity of first immediate release core 52. Still further, dosage form 60 shown in FIG. 6 comprises a first immediate release core 62 and osmotic chamber 10 that are surrounded or embedded in shell materials having distinct portions 66 and 68 such that first shell material 66 in the vicinity of first immediate release core 62 has immediate release properties, while second shell material 68 provided over osmotic chamber 10 has a modified release profile.

The active ingredient or ingredients are present in the dosage form in a therapeutically effective amount, which is an amount that produces the desired therapeutic response upon oral administration and can be readily determined by one skilled in the art. In determining such amounts, the particular active ingredient being administered, the bioavailability characteristics of the active ingredient, the dosing regimen, the age and weight of the patient, and other factors must be considered, as known in the art. Typically, the dosage form comprises at least about 1 weight percent, for example, the dosage form comprises at least about 5 weight percent, say at least about 20 weight percent of a combination of one or more active ingredients. In one embodiment, a core comprises a total of at least about 25 weight percent (based on the weight of the core) of one or more active ingredients.

The active ingredient or ingredients may be present in the dosage form in any form. For example, the active ingredient may be dispersed at the molecular level, e.g. melted or dissolved, within the dosage form, or may be in the form of particles, which in turn may be coated or uncoated. If an active ingredient is in the form of particles, the particles (whether coated or uncoated) typically have an average particle size of about 1-2000 microns.

Each core may be any solid form. As used herein, “core” refers to a material that is at least partially enveloped or surrounded by another material. Preferably, a core is a self-contained unitary object, such as a tablet or capsule. Typically, a core comprises a solid, for example, a core may be a compressed or molded tablet, hard or soft capsule, suppository, or a confectionery form such as a lozenge, nougat, caramel, fondant, or fat based composition. In certain other embodiments, a core or a portion thereof may be in the form of a semi-solid or a liquid in the finished dosage form. For example a core may comprise a liquid filled capsule, or a semisolid fondant material. In embodiments in which a core comprises a flowable component, such as a plurality of granules or particles, or a liquid, the core preferably additionally comprises an enveloping component, such as a capsule shell, or a coating, for containing the flowable material. In certain particular embodiments in which a core comprises an enveloping component, the shell or shell portions of the present invention are in direct contact with the enveloping component of the core, which separates the shell from the flowable component of the core.

The core may have one of a variety of different shapes. Each core may have the same or different physical dimensions, shape, etc. as the other cores. In certain embodiments, a core has one or more major faces. For example, in embodiments wherein a core is a compressed tablet, the core surface typically has opposing upper and lower faces formed by contact with the upper and lower punch faces in the compression machine. In such embodiments the core surface typically further comprises a “belly-band” located between the upper and lower faces, and formed by contact with the die walls in the compression machine. A core may also comprise a multilayer tablet.

In one embodiment at least one core is a compressed tablet having a hardness from about 2 to about 30 kp/cm², e.g. from about 6 to about 25 kp/cm². “Hardness” is a term used in the art to describe the diametral breaking strength of either the core or the coated solid dosage form as measured by conventional pharmaceutical hardness testing equipment, such as a Schleuniger Hardness Tester. In order to compare values across different size tablets, the breaking strength must be normalized for the area of the break. This normalized value, expressed in kp/cm², is sometimes referred in the art as tablet tensile strength. A general discussion of tablet hardness testing is found in Leiberman et al., Pharmaceutical Dosage Forms—Tablets, Volume 2, 2^{nd} ed., Marcel Dekker Inc., 1990, pp. 213-217, 327-329. In another embodiment, all the cores in the dosage form comprise a compressed tablet having a hardness from about 2 to about 30 kp/cm², e.g. from about 6 to about 25 kp/cm².

Exemplary core shapes that may be employed include tablet shapes formed from compression tooling shapes described by “The Elizabeth Companies Tablet Design Training Manual” (Elizabeth Carbide Die Co., Inc., p. 7 (McKeesport, Pa.) (incorporated herein by reference). The cores may be prepared by any suitable method, including for example compression or molding, and depending on the method by which they are made, typically comprise active ingredient and a variety of excipients.

In embodiments in which one or more cores, or portions thereof are made by compression, suitable excipients include fillers, binders, disintegrants, lubricants, glidants, and the like, as known in the art. In embodiments in which a core is made by compression and additionally confers modified release of an active ingredient contained therein, such core preferably further comprises a release-modifying compressible excipient.

Suitable fillers for use in making a core or core portion by compression include water-soluble compressible carbohydrates such as sugars, which include dextrose, sucrose, maltose, and lactose, sugar-alcohols, which include mannitol, sorbitol, maltitol, xylitol, starch hydrolysates, which include dextrins, and maltodextrins, and the like, water insoluble plastically deforming materials such as microcrystalline cellulose or other cellulosic derivatives, water-insoluble brittle fracture materials such as dicalcium phosphate, tricalcium phosphate and the like and mixtures thereof.

Suitable binders for making a core or core portion by compression include dry binders such as polyvinyl pyrrolidone, hydroxypropylmethylcellulose, and the like; wet binders such as water-soluble polymers, including hydrocolloids such as acacia, alginates, agar, guar gum, locust bean, carrageenan, carboxymethylcellulose, tara, gum arabic, tragacanth, pectin, xanthan, gellan, gelatin, maltodextrin, galactomannan, pusstulan, laminarin, scleroglucan, inulin, whelan, rhamsan, zooglan, methylan, chitin, cyclodextrin, chitosan, polyvinyl pyrrolidone, cellulosics, sucrose, starches, and the like; and derivatives and mixtures thereof.

Suitable disintegrants for making a core or core portion by compression, include sodium starch glycolate, cross-linked polyvinylpyrrolidone, cross-linked carboxymethylcellulose, starches, microcrystalline cellulose, and the like. Suitable lubricants for making a core or core portion by compression include long chain fatty acids and their salts, such as magnesium stearate and stearic acid, talc, glycerides and waxes. Suitable glidants for making a core or core portion by compression, include colloidal silicon dioxide, and the like. Suitable release-modifying excipients for making a core or core portion by compression include swellable erodible hydrophilic materials, insoluble edible materials, pH-dependent polymers, and the like.

Suitable swellable erodible hydrophilic materials for use as release-modifying excipients for making a core or core portion by compression include: water swellable cellulose derivatives, polyalkylene glycols, thermoplastic polyalkylene oxides, acrylic polymers, hydrocolloids, clays, gelling starches, and swelling cross-linked polymers, and derivatives, copolymers, and combinations thereof. Examples of suitable water swellable cellulose derivatives include sodium carboxymethylcellulose, cross-linked hydroxypropylcellulose, hydroxypropyl cellulose (HPC), hydroxypropylmethylcellulose (HPMC), hydroxyisopropylcellulose, hydroxybutylcellulose, hydroxyphenylcellulose, hydroxyethylcellulose (HEC), hydroxypentylcellulose, hydroxypropylethylcellulose, hydroxypropylbutylcellulose, hydroxypropylethylcellulose. Examples of suitable polyalkylene glycols include polyethylene glycol. Examples of suitable thermoplastic polyalkylene oxides include poly (ethylene oxide). Examples of suitable acrylic polymers include potassium methacrylate divinylbenzene copolymer, polymethylmethacrylate, CARBOPOL (high-molecular weight cross-linked acrylic acid homopolymers and copolymers), and the like. Examples of suitable hydrocolloids include alginates, agar, guar gum, locust bean gum, kappa carrageenan, iota carrageenan, tara, gum arabic, tragacanth, pectin, xanthan gum, gellan gum, maltodextrin, galactomannan, pusstulan, laminarin, scleroglucan, gum arabic, inulin, pectin, gelatin, whelan, rhamsan, zooglan, methylan, chitin, cyclodextrin, chitosan. Examples of suitable clays include smectites such as bentonite, kaolin, and laponite; magnesium trisilicate, magnesium aluminum silicate, and the like, and derivatives and mixtures thereof. Examples of suitable gelling starches include acid hydrolyzed starches, swelling starches such as sodium starch glycolate, and derivatives thereof. Examples of suitable swelling cross-linked polymers include cross-linked polyvinyl pyrrolidone, cross-linked agar, and cross-linked carboxymethylcellulose sodium.

Suitable insoluble edible materials for use as release-modifying excipients for making a core or core portion by compression include water-insoluble polymers, and low-melting hydrophobic materials. Examples of suitable water-insoluble polymers include ethylcellulose, polyvinyl alcohols, polyvinyl acetate, polycaprolactones, cellulose acetate and its derivatives, acrylates, methacrylates, acrylic acid copolymers; and the like and derivatives, copolymers, and combinations thereof. Suitable low-melting hydrophobic materials include fats, fatty acid esters, phospholipids, and waxes. Examples of suitable fats include hydrogenated vegetable oils such as for example cocoa butter, hydrogenated palm kernel oil, hydrogenated cottonseed oil, hydrogenated sunflower oil, and hydrogenated soybean oil; and free fatty acids and their salts. Examples of suitable fatty acid esters include sucrose fatty acid esters, mono, di, and triglycerides, glyceryl behenate, glyceryl palmitostearate, glyceryl monostearate, glyceryl tristearate, glyceryl trilaurylate, glyceryl myristate, GlycoWax-932, lauroyl macrogol-32 glycerides, and stearoyl macrogol-32 glycerides. Examples of suitable phospholipids include phosphotidyl choline, phosphotidyl serene, phosphotidyl enositol, and phosphotidic acid. Examples of suitable waxes include carnauba wax, spermaceti wax, beeswax, candelilla wax, shellac wax, microcrystalline wax, and paraffin wax; fat-containing mixtures such as chocolate; and the like.

Suitable pH-dependent polymers for use as release-modifying excipients for making a core or core portion by compression include enteric cellulose derivatives, for example hydroxypropyl methylcellulose phthalate, hydroxypropyl methylcellulose acetate succinate, cellulose acetate phthalate; natural resins such as shellac and zein; enteric acetate derivatives such as for example polyvinylacetate phthalate, cellulose acetate phthalate, acetaldehyde dimethylcellulose acetate; and enteric acrylate derivatives such as for example polymethacrylate-based polymers such as poly(methacrylic acid, methyl methacrylate) 1:2, which is commercially available from Rohm Pharma GmbH under the tradename EUDRAGIT S, and poly(methacrylic acid, methyl methacrylate) 1:1, which is commercially available from Rohm Pharma GmbH under the tradename EUDRAGIT L, and the like, and derivatives, salts, copolymers, and combinations thereof.

Suitable pharmaceutically acceptable adjuvants for making a core or core portion by compression include, preservatives; high intensity sweeteners such as aspartame, acesulfame potassium, sucralose, and saccharin; flavorants; colorants; antioxidants; surfactants; wetting agents; and the like and mixtures thereof.

In embodiments wherein one or more cores are prepared by compression, a dry blending (i.e. direct compression), or wet granulation process may be employed, as known in the art. In a dry blending (direct compression) method, the active ingredient or ingredients, together with the excipients, are blended in a suitable blender, than transferred directly to a compression machine for pressing into tablets. In a wet granulation method, the active ingredient or ingredients, appropriate excipients, and a solution or dispersion of a wet binder (e.g. an aqueous cooked starch paste, or solution of polyvinyl pyrrolidone) are mixed and granulated. Alternatively a dry binder may be included among the excipients, and the mixture may be granulated with water or other suitable solvent. Suitable apparatuses for wet granulation are known in the art, including low shear, e.g. planetary mixers; high shear mixers; and fluid beds, including rotary fluid beds. The resulting granulated material is dried and optionally dry-blended with further ingredients, e.g. adjuvants and/or excipients such as for example lubricants, colorants, and the like. The final dry blend is then suitable for compression. Methods for direct compression and wet granulation processes are known in the art, and are described in detail in, for example, Lachman, et al., The Theory and Practice of Industrial Pharmacy, Chapter 11 (3^{rd} ed. 1986).

The dry-blended, or wet granulated, powder mixture is typically compacted into tablets using a rotary compression machine as known in the art, such as for example those commercially available from Fette America Inc., Rockaway, N.J., or Manesty Machines LTD, Liverpool, UK. In a rotary compression machine, a metered volume of powder is filled into a die cavity, which rotates as part of a “die table” from the filling position to a compaction position where the powder is compacted between an upper and a lower punch to an ejection position where the resulting tablet is pushed from the die cavity by the lower punch and guided to an ejection chute by a stationary “take-off” bar.

In one embodiment, at least one core is prepared by the compression methods and apparatus described in copending U.S. Pat. No. 6,767,200, the disclosure of which is incorporated herein by reference. Specifically, the core is made using a rotary compression module comprising a fill zone, compression zone, and ejection zone in a single apparatus having a double row die construction as shown in FIG. 6 of U.S. Pat. No. 6,767,200. The dies of the compression module are preferably filled using the assistance of a vacuum, with filters located in or near each die.

Immediate release and osmotic cores made by compression may be single or multi-layer, for example bi-layer, tablets.

In certain embodiments the dosage form comprises multiple shell portions that are compositionally and/or functionally different. As used herein, the term “compositionally different” means having features that are readily distinguishable by qualitative or quantitative chemical analysis, physical testing, or visual observation. For example, the first and second shell portions may contain different ingredients, or different levels of the same ingredients, or the first and second shell portions may have different physical or chemical properties, different functional properties, or be visually distinct. Examples of physical or chemical properties that may be different include hydrophylicity, hydrophobicity, hygroscopicity, elasticity, plasticity, tensile strength, crystallinity, and density. Examples of functional properties which may be different include rate and/or extent of dissolution of the material itself or of an active ingredient therefrom, rate of disintegration of the material, permeability to active ingredients, permeability to water or aqueous media, and the like. Examples of visual distinctions include size, shape, topography, or other geometric features, color, hue, opacity, and gloss.

The use of subcoatings is well known in the art and disclosed in, for example, U.S. Pat. No. 3,185,626, which is incorporated by reference herein. Any composition suitable for film-coating a tablet may be used as a subcoating according to the present invention. Examples of suitable subcoatings are disclosed in U.S. Pat. Nos. 4,683,256, 4,543,370, 4,643,894, 4,828,841, 4,725,441, 4,802,924, 5,630,871, and 6,274,162, which are all incorporated by reference herein. Additional suitable subcoatings include one or more of the following ingredients: cellulose ethers such as hydroxypropylmethylcellulose, hydroxypropylcellulose, and hydroxyethylcellulose; polycarbohydrates such as xanthan gum, starch, and maltodextrin; plasticizers including for example, glycerin, polyethylene glycol, propylene glycol, dibutyl sebecate, triethyl citrate, vegetable oils such as castor oil, surfactants such as Polysorbate-80, sodium lauryl sulfate and dioctyl-sodium sulfosuccinate; polycarbohydrates, pigments, and opacifiers.

The dosage forms of the invention provide modified release of one or more active ingredients contained therein. The active ingredient or ingredients may be found within one or more cores, the osmotic chamber(s), the shell, or portions or combinations thereof. Preferably, one or more active ingredients are contained in one or more cores and osmotic chamber(s). More preferably, at least one active ingredient is contained in each of the immediate release core and osmotic chamber.

The shell, or a portion thereof can provide for a modified release of at least one active ingredient in the dosage form. As used herein, the term “modified release” means the release of an active ingredient from a dosage form or a portion thereof in other than an immediate release fashion, i.e., other than immediately upon contact of the dosage form or portion thereof with a liquid medium. As known in the art, types of modified release include delayed or controlled. Types of controlled release include prolonged, sustained, extended, retarded, and the like. Modified release profiles that incorporate a delayed release feature include pulsatile, repeat action, and the like. As is also known in the art, suitable mechanisms for achieving modified release of an active ingredient include diffusion, erosion, surface area control via geometry and/or impermeable barriers, and other known mechanisms known.

In a preferred embodiment, at least one active ingredient is released from the first core in an immediate release fashion. As used herein, “immediate release” means the dissolution characteristics of an active ingredient meets USP specifications for immediate release tablets containing the active ingredient. For example, for acetaminophen tablets, USP 24 specifies that in pH 5.8 phosphate buffer, using USP apparatus 2 (paddles) at 50 rpm, at least 80% of the acetaminophen contained in the dosage form is released therefrom within 30 minutes after dosing, and for ibuprofen tablets, USP 24 specifies that in pH 7.2 phosphate buffer, using USP apparatus 2 (paddles) at 50 rpm, at least 80% of the ibuprofen contained in the dosage form is released therefrom within 60 minutes after dosing. See USP 24, 2000 Version, 19-20 and 856 (1999).

The composition of the shell may function to modify the release therethrough of an active ingredient contained in an underlying core. In one embodiment, the shell may function to delay release of an active ingredient from an underlying core. In another embodiment, the shell may function to sustain, extend, retard, or prolong the release of at least one active ingredient from the second (distally located) core. In one embodiment the immediate release of the active ingredient in the first core is provided by one or more openings in the shell suitable to provide immediate release. A plurality of openings having a diameter of least 1 mm (1000 microns) produces the desired flow characteristics. In another embodiment, the immediate release of the active ingredient in the first core is provided through the shape of the core. For example, a core can be provided in a position to cause the shell to rupture by ensuring that the shell is thinner on the side of the immediate release core. The thickness of the coating suitable for rupture is less than 1 mm (1000 microns), preferably less than 500 microns. In another embodiment the immediate release of the first core is provided through the addition of a water soluble fill material embedded in one portion of the shell in proximity to the immediate release core.

In one embodiment, the shell comprises a release modifying moldable excipient, such as, but not limited to, swellable erodible hydrophilic materials described above.

In another embodiment, the dosage form is substantially free (i.e. less than 1% by weight, preferably less than about 0.1% by weight, based upon the shell weight) of charge control agents. As used herein, the term “charge control agents” refers to a material having a charge control function, such as those used for electrostatic deposition of coatings onto substrates. Such charge control agents include metal salicylates, for example zinc salicylate, magnesium salicylate and calcium salicylate; quaternary ammonium salts; benzalkonium chloride; benzethonium chloride; trimethyl tetradecyl ammonium bromide (cetrimide); and cyclodextrins and their adducts.

In a second preferred embodiment such as described in the preceding paragraphs, one or more active ingredients contained in the second core are released in a controlled, sustained, prolonged, or extended manner beginning initially upon contact of the dosage for with a liquid medium, with or without a substantial preceding lag time, e.g. release of at least one active ingredients begins within 30 minutes, e.g. within 15 minutes, say within 10 minutes, of contact of the dosage form with a liquid medium.

In certain embodiments, the shell itself, e.g. a portion thereof, or an outer coating thereon may also contain active ingredient. In one embodiment, such active ingredient will be released immediately from the dosage form upon ingestion, or contacting of the dosage form with a liquid medium. In another embodiment, such active ingredient will be released in a controlled, sustained, prolonged, or extended fashion upon ingestion, or contacting of the dosage form with a liquid medium.

In certain preferred embodiments of the invention, the cores, the shell, the fill material or any portions thereof, or all are prepared by molding. In particular, the cores, the shell, the fill material or all may be made by solvent-based molding or solvent-free molding. In such embodiments, the core, the shell, the fill material or all are made from a flowable material optionally comprising active ingredient. The flowable material may be any edible material that is flowable at a temperature between about 37° C. and 250° C., and that is solid, semi-solid, or can form a gel at a temperature between about −10° C. and about 35° C. When it is in the fluid or flowable state, the flowable material may comprise a dissolved or molten component for solvent-free molding, or optionally a solvent such as for example water or organic solvents, or combinations thereof, for solvent-based molding. The solvent may be partially or substantially removed by drying.

In one embodiment, solvent-based or solvent-free molding is performed to produce the core and/or shell via thermal setting molding using the method and apparatus described in published U.S. patent application 2003-0124183 A1, the disclosure of which is incorporated herein by reference. In this embodiment, a core a shell, a fill material or all are formed by injecting flowable form into a molding chamber. The flowable material preferably comprises a thermal setting material at a temperature above its melting point but below the decomposition temperature of any active ingredient contained therein. The flowable material is cooled and solidifies in the molding chamber into a shaped form (i.e., having the shape of the mold).

According to this method, the flowable material may comprise solid particles suspended in a molten matrix, for example a polymer matrix. The flowable material may be completely molten or in the form of a paste. The flowable material may comprise an active ingredient dissolved in a molten material in the case of solvent-free molding. Alternatively, the flowable material may be made by dissolving a solid in a solvent, which solvent is then evaporated after the molding step in the case of solvent-based molding.

In the thermal cycle molding method and apparatus of published U.S. patent application US 2003-0086973 A1, the disclosure of which is incorporated herein by reference, a thermal cycle molding module having the general configuration shown in FIG. 3 therein is employed. The thermal cycle molding module comprises a rotor around which a plurality of mold units are disposed. The thermal cycle molding module includes a reservoir for holding flowable material to make the core. In addition, the thermal cycle molding module is provided with a temperature control system for rapidly heating and cooling the mold units.

The mold units may comprise center mold assemblies, upper mold assemblies, and lower mold assemblies that mate to form mold cavities having a desired shape, for instance of a core or a shell surrounding one or more cores. As rotor rotates, opposing center and upper mold assemblies or opposing center and lower mold assemblies close. Flowable material, which is heated to a flowable state in reservoir, is injected into the resulting mold cavities. The temperature of the flowable material is then decreased, hardening the flowable material. The mold assemblies open and eject the finished product.

In one embodiment of the invention, the shell or fill material is applied to the dosage form using a thermal cycle molding apparatus of the general type of published U.S. application US 2003-0086973 A1 comprising rotatable center mold assemblies, lower mold assemblies and upper mold assemblies. Cores and osmotic chambers are continuously fed to the mold assemblies. Shell or fill flowable material, which is heated to a flowable state in reservoir, is injected into the mold cavities created by the closed mold assemblies holding the cores. The temperature of the shell or fill flowable material is then decreased, hardening it around the cores and osmotic chambers. The mold assemblies open and eject the finished dosage forms. Shell coating is performed in two steps, each half of the dosage forms being coated separately as shown in the flow diagram published U.S. patent application 2003-0068367 A1 via rotation of the center mold assembly.

In particular, the mold assemblies for applying the shell are provided with two or more cavities to accommodate the desired number of cores in the dosage form. A wall, preferably made of rubber or metal, separates the cavities and the overall shape of the cavities conform to the shape of the cores.

In one embodiment of the invention, the shell is applied to the dosage form using a zero cycle molding apparatus of the general type of published U.S. application U.S. Ser. No. ______, MCP5018 (Ser. No. 10/677,984) comprising rotatable center mold assemblies, lower mold assemblies and upper mold assemblies. Cores and osmotic chambers are continuously fed to the mold assemblies. Shell flowable material, which is heated to a flowable state in reservoir, is injected into the mold cavities created by the closed mold assemblies holding the cores and osmotic chambers. The mold assemblies open and eject the finished dosage forms. Shell coating is preferably performed in two steps, each half of the dosage forms being coated separately via rotation of the center mold assembly.

In particular, the mold assemblies for applying the shell are provided with two or more cavities to accommodate the desired number of cores and osmotic chambers in the dosage form. A wall, preferably made of rubber or metal, separates the cavities and the overall shape of the cavities conform to the shape of the cores.

In one embodiment, the compression module of U.S. Pat. No. 6,767,200 may be employed to make cores. The shell may be made applied to these cores and osmotic chambers using a thermal cycle molding module as described above. A transfer device may be used to transfer the cores and osmotic chambers from the compression module to the molding module. Such a transfer device may have the structure shown published U.S. patent application 2003-0068367 A1. The device comprises a plurality of transfer units attached in cantilever fashion to a belt. The transfer device rotates and operates in sync with the compression module and the molding module to which it is coupled. Transfer units comprise retainers for holding cores and osmotic chambers as they travel around the transfer device.

Each transfer unit comprises multiple retainers for holding multiple cores and osmotic chambers side by side. In one embodiment, the space between the retainers within each transfer unit is adjusted via a cam track/cam follower mechanism as the transfer units move around the transfer device. On arrival at the selected molding module, the cores and osmotic chambers are grouped together for placement in a single dosage form, which have been held within a single transfer unit, are properly spaced from one another and ready to be fed into the mold assemblies. The cores may comprise a single layer or multiple layers.

Suitable thermoplastic materials for use in or as the flowable material include both water-soluble and water insoluble polymers that are generally linear, not crosslinked, and not strongly hydrogen bonded to adjacent polymer chains. Examples of suitable thermoplastic materials include: thermoplastic water-swellable cellulose derivatives, thermoplastic water insoluble cellulose derivatives, thermoplastic vinyl polymers, thermoplastic starches, thermoplastic polyalkylene glycols, thermoplastic polyalkylene oxides, and amorphous sugar-glass, and the like, and derivatives, copolymers, and combinations thereof. Examples of suitable thermoplastic water swellable cellulose derivatives include hydroxypropyl cellulose (HPC), hydroxypropylmethyl cellulose (HPMC), methyl cellulose (MC). Examples of suitable thermoplastic water insoluble cellulose derivatives include cellulose acetate (CA), ethyl cellulose (EC), cellulose acetate butyrate (CAB), cellulose propionate. Examples of suitable thermoplastic vinyl polymers include polyvinyl alcohol (PVA) and polyvinyl pyrrolidone (PVP). Examples of suitable thermoplastic starches are disclosed for example in U.S. Pat. No. 5,427,614. Examples of suitable thermoplastic polyalkylene glycols include polyethylene glycol. Examples of suitable thermoplastic polyalkylene oxides include polyethylene oxide having a molecular weight from about 100,000 to about 900,000 Daltons. Other suitable thermoplastic materials include sugar in the form on an amorphous glass such as that used to make hard candy forms.

Any film former known in the art is suitable for use in the flowable material. Examples of suitable film formers include, but are not limited to, film-forming water-soluble polymers, film-forming proteins, film-forming water insoluble polymers, and film-forming pH-dependent polymers. In one embodiment, the film-former for making the core or shell or portion thereof by molding may be selected from cellulose acetate, ammonium methacrylate copolymer type B, shellac, hydroxypropylmethylcellulose, and polyethylene oxide, and combinations thereof.

Suitable film-forming water soluble polymers include water soluble vinyl polymers such as polyvinyl alcohol (PVA); water soluble polycarbohydrates such as hydroxypropyl starch, hydroxyethyl starch, pullulan, methylethyl starch, carboxymethyl starch, pre-gelatinized starches, and film-forming modified starches; water swellable cellulose derivatives such as hydroxypropyl cellulose (HPC), hydroxypropylmethyl cellulose (HPMC), methyl cellulose (MC), hydroxyethylmethylcellulose (HEMC), hydroxybutylmethylcellulose (HBMC), hydroxyethylethylcellulose (HEEC), and hydroxyethylhydroxypropylmethyl cellulose (HEMPMC); water soluble copolymers such as methacrylic acid and methacrylate ester copolymers, polyvinyl alcohol and polyethylene glycol copolymers, polyethylene oxide and polyvinylpyrrolidone copolymers; and derivatives and combinations thereof.

Suitable film-forming proteins may be natural or chemically modified, and include gelatin, whey protein, myofibrillar proteins, coagulatable proteins such as albumin, casein, caseinates and casein isolates, soy protein and soy protein isolates, zein; and polymers, derivatives and mixtures thereof.

Suitable film-forming water insoluble polymers, include for example ethylcellulose, polyvinyl alcohols, polyvinyl acetate, polycaprolactones, cellulose acetate and its derivatives, acrylates, methacrylates, acrylic acid copolymers; and the like and derivatives, copolymers, and combinations thereof.

Suitable film-forming pH-dependent polymers include enteric cellulose derivatives, such as for example hydroxypropyl methylcellulose phthalate, hydroxypropyl methylcellulose acetate succinate, cellulose acetate phthalate; natural resins, such as shellac and zein; enteric acetate derivatives such as for example polyvinylacetate phthalate, cellulose acetate phthalate, acetaldehyde dimethylcellulose acetate; and enteric acrylate derivatives such as for example polymethacrylate-based polymers such as poly(methacrylic acid, methyl methacrylate) 1:2, which is commercially available from Rohm Pharma GmbH under the tradename, EUDRAGIT S, and poly(methacrylic acid, methyl methacrylate) 1:1, which is commercially available from Rohm Pharma GmbH under the tradename, EUDRAGIT L, and the like, and derivatives, salts, copolymers, and combinations thereof.

One suitable hydroxypropylmethylcellulose compound for use as a thermoplastic film-forming water soluble polymer is “HPMC 2910”, which is a cellulose ether having a degree of substitution of about 1.9 and a hydroxypropyl molar substitution of 0.23, and containing, based upon the total weight of the compound, from about 29% to about 30% methoxyl groups and from about 7% to about 12% hydroxylpropyl groups. HPMC 2910 is commercially available from the Dow Chemical Company under the tradename METHOCEL E. METHOCEL E5, which is one grade of HPMC-2910 suitable for use in the present invention, has a viscosity of about 4 to 6 cps (4 to 6 millipascal-seconds) at 20° C. in a 2% aqueous solution as determined by a Ubbelohde viscometer. Similarly, METHOCEL E6, which is another grade of HPMC-2910 suitable for use in the present invention, has a viscosity of about 5 to 7 cps (5 to 7 millipascal-seconds) at 20° C. in a 2% aqueous solution as determined by a Ubbelohde viscometer. METHOCEL El5, which is another grade of HPMC-2910 suitable for use in the present invention, has a viscosity of about 15000 cps (15 millipascal-seconds) at 20° C. in a 2% aqueous solution as determined by a Ubbelohde viscometer. As used herein, “degree of substitution” means the average number of substituent groups attached to an anhydroglucose ring, and “hydroxypropyl molar substitution” means the number of moles of hydroxypropyl per mole anhydroglucose.

One suitable polyvinyl alcohol and polyethylene glycol copolymer is commercially available from BASF Corporation under the tradename KOLLICOAT IR.

As used herein, “modified starches” include starches that have been modified by is crosslinking, chemically modified for improved stability or optimized performance, or physically modified for improved solubility properties or optimized performance. Examples of chemically modified starches are well known in the art and typically include those starches that have been chemically treated to cause replacement of some of its hydroxyl groups with either ester or ether groups. Crosslinking, as used herein, may occur in modified starches when two hydroxyl groups on neighboring starch molecules are chemically linked. As used herein, “pre-gelatinized starches” or “instantized starches” refers to modified starches that have been pre-wetted, then dried to enhance their cold-water solubility. Suitable modified starches are commercially available from several suppliers such as, for example, A.E. Staley Manufacturing Company, and National Starch & Chemical Company. One suitable film forming modified starch includes the pre-gelatinized waxy maize derivative starches that are commercially available from National Starch & Chemical Company under the tradenames PURITY GUM and FILMSET, and derivatives, copolymers, and mixtures thereof. Such waxy maize starches typically contain, based upon the total weight of the starch, from about 0 percent to about 18 percent of amylose and from about 100% to about 88% of amylopectin.

Other suitable film forming modified starches include the hydroxypropylated starches, in which some of the hydroxyl groups of the starch have been etherified with hydroxypropyl groups, usually via treatment with propylene oxide. One example of a suitable hydroxypropyl starch that possesses film-forming properties is available from Grain Processing Company under the tradename, PURE-COTE B790.

Suitable tapioca dextrins for use as film formers include those available from National Starch & Chemical Company under the tradenames CRYSTAL GUM or K-4484, and derivatives thereof such as modified food starch derived from tapioca, which is available from National Starch and Chemical under the tradename PURITY GUM 40, and copolymers and mixtures thereof.

Any thickener known in the art is suitable for use in the flowable material of the present invention. Examples of such thickeners include but are not limited to hydrocolloids (also referred to herein as gelling polymers), clays, gelling starches, and crystallizable carbohydrates, and derivatives, copolymers and mixtures thereof.

Examples of suitable hydrocolloids (also referred to herein as gelling polymers) such as alginates, agar, guar gum, locust bean, carrageenan, tara, gum arabic, tragacanth, pectin, xanthan, gellan, maltodextrin, galactomannan, pusstulan, laminarin, scleroglucan, gum arabic, inulin, pectin, whelan, rhamsan, zooglan, methylan, chitin, cyclodextrin, chitosan. Examples of suitable clays include smectites such as bentonite, kaolin, and laponite; magnesium trisilicate, magnesium aluminum silicate, and the like, and derivatives and mixtures thereof. Examples of suitable gelling starches include acid hydrolyzed starches, and derivatives and mixtures thereof. Additional suitable thickening hydrocolloids include low-moisture polymer solutions such as mixtures of gelatin and other hydrocolloids at water contents up to about 30%, such as for example those used to make “gummy” confection forms.

Additional suitable thickeners include crystallizable carbohydrates, and the like, and derivatives and combinations thereof. Suitable crystallizable carbohydrates include the monosaccharides and the oligosaccharides. Of the monosaccharides, the aldohexoses e.g., the D and L isomers of allose, altrose, glucose, mannose, gulose, idose, galactose, talose, and the ketohexoses e.g., the D and L isomers of fructose and sorbose along with their hydrogenated analogs: e.g., glucitol (sorbitol), and mannitol are preferred. Of the oligosaccharides, the 1,2-disaccharides sucrose and trehalose, the 1,4-disaccharides maltose, lactose, and cellobiose, and the 1,6-disaccharides gentiobiose and melibiose, as well as the trisaccharide raffinose are preferred along with the isomerized form of sucrose known as isomaltulose and its hydrogenated analog isomalt. Other hydrogenated forms of reducing disaccharides (such as maltose and lactose), for example, maltitol and lactitol are also preferred. Additionally, the hydrogenated forms of the aldopentoses: e.g., D and L ribose, arabinose, xylose, and lyxose and the hydrogenated forms of the aldotetroses: e.g., D and L erythrose and throse are preferred and are exemplified by xylitol and erythritol, respectively.

In one embodiment of the invention, the flowable material comprises gelatin as a gelling polymer. Gelatin is a natural, thermogelling polymer. It is a tasteless and colorless mixture of derived proteins of the albuminous class that is ordinarily soluble in warm water. Two types of gelatin—Type A and Type B—are commonly used. Type A gelatin is a derivative of acid-treated raw materials. Type B gelatin is a derivative of alkali-treated raw materials. The moisture content of gelatin, as well as its Bloom strength, composition and original gelatin processing conditions, determine its transition temperature between liquid and solid. Bloom is a standard measure of the strength of a gelatin gel, and is roughly correlated with molecular weight. Bloom is defined as the weight in grams required to move a half-inch diameter plastic plunger 4 mm into a 6.67% gelatin gel that has been held at 10° C. for 17 hours. In a preferred embodiment, the flowable material is an aqueous solution comprising 20% 275 Bloom pork skin gelatin, 20% 250 Bloom Bone Gelatin, and approximately 60% water.

Suitable xanthan gums include those available from C.P. Kelco Company under the tradenames KELTROL 1000, XANTROL 180, or K9B310.

Suitable clays include smectites such as bentonite, kaolin, and laponite; magnesium trisilicate, magnesium aluminum silicate, and the like, and derivatives and mixtures thereof.

“Acid-hydrolyzed starch,” as used herein, is one type of modified starch that results from treating a starch suspension with dilute acid at a temperature below the gelatinization point of the starch. During the acid hydrolysis, the granular form of the starch is maintained in the starch suspension, and the hydrolysis reaction is ended by neutralization, filtration and drying once the desired degree of hydrolysis is reached. As a result, the average molecular size of the starch polymers is reduced. Acid-hydrolyzed starches (also known as “thin boiling starches”) tend to have a much lower hot viscosity than the same native starch as well as a strong tendency to gel when cooled.

“Gelling starches,” as used herein, include those starches that, when combined with water and heated to a temperature sufficient to form a solution, thereafter form a gel upon cooling to a temperature below the gelation point of the starch. Examples of gelling starches include, but are not limited to, acid hydrolyzed starches such as that available from Grain Processing Corporation under the tradename PURE-SET B950; hydroxypropyl distarch phosphate such as that available from Grain Processing Corporation under the tradename, PURE-GEL B990, and mixtures thereof.

Suitable low-melting hydrophobic materials include fats, fatty acid esters, phospholipids, and waxes. Examples of suitable fats include hydrogenated vegetable oils such as for example cocoa butter, hydrogenated palm kernel oil, hydrogenated cottonseed oil, hydrogenated sunflower oil, and hydrogenated soybean oil; and free fatty acids and their salts. Examples of suitable fatty acid esters include sucrose fatty acid esters, mono, di, and triglycerides, glyceryl behenate, glyceryl palmitostearate, glyceryl monostearate, glyceryl tristearate, glyceryl trilaurylate, glyceryl myristate, GlycoWax-932, lauroyl macrogol-32 glycerides, and stearoyl macrogol-32 glycerides. Examples of suitable phospholipids include phosphotidyl choline, phosphotidyl serene, phosphotidyl enositol, and phosphotidic acid. Examples of suitable waxes include carnauba wax, spermaceti wax, beeswax, candelilla wax, shellac wax, microcrystalline wax, and paraffin wax; fat-containing mixtures such as chocolate; and the like.

Suitable non-crystallizable carbohydrates include non-crystallizable sugars such as polydextrose, and starch hydrolysates, e.g. glucose syrup, corn syrup, and high fructose corn syrup; and non-crystallizable sugar-alcohols such as maltitol syrup.

Suitable solvents for optional use as components of the flowable material for making the core or the shell by molding include water; polar organic solvents such as methanol, ethanol, isopropanol, acetone, and the like; and non-polar organic solvents such as methylene chloride, and the like; and mixtures thereof.

The flowable material for making cores, the shell, or fill material by molding may optionally comprise adjuvants or excipients, which may comprise up to about 30% by weight of the flowable material. Examples of suitable adjuvants or excipients include plasticizers, detackifiers, humectants, surfactants, anti-foaming agents, colorants, flavorants, sweeteners, opacifiers, and the like. Suitable plasticizers for making the core, the shell, or a portion thereof, by molding include, but not be limited to polyethylene glycol; propylene glycol; glycerin; sorbitol; triethyl citrate; tributyl citrate; dibutyl sebecate; vegetable oils such as castor oil, rape oil, olive oil, and sesame oil; surfactants such as Polysorbates, sodium lauryl sulfates, and dioctyl-sodium sulfosuccinates; mono acetate of glycerol; diacetate of glycerol; triacetate of glycerol; natural gums; triacetin; acetyltributyl citrate; diethyloxalate; diethylmalate; diethyl fumarate; diethylmalonate; dioctylphthalate; dibutylsuccinate; glyceroltributyrate; hydrogenated castor oil; fatty acids; substituted triglycerides and glycerides; and the like and/or mixtures thereof. In one embodiment, the plasticizer is triethyl citrate. In certain embodiments, the shell is substantially free of plasticizers, i.e. contains less than about 1%, say less than about 0.01% of plasticizers.

In embodiments in which the shell is prepared using a solvent-free molding process, the shell typically comprises at least about 30 percent, e.g. at least about 45 percent by weight of a thermal-reversible carrier. The shell may optionally further comprise up to about 55 weight percent of a release-modifying excipient. The shell may optionally further comprise up to about 30 weight percent total of various plasticizers, adjuvants and excipients. In certain embodiments in which the shell is prepared by solvent-free molding, and functions to delay the release of one or more active ingredients from an underlying core, the release modifying excipient is preferably selected from swellable, erodible hydrophilic materials.

In embodiments in which the shell is prepared using a solvent-based molding process, the shell typically comprises at least about 10 weight percent, e.g. at least about 12 weight percent or at least about 15 weight percent or at least about 20 weight percent or at least about 25 weight percent of a film-former. Here, the shell may optionally further comprise up to about 55 weight percent of a release-modifying excipient. The shell may again also optionally further comprise up to about 30 weight percent total of various plasticizers, adjuvants, and excipients.

In embodiments in which the shell is applied to the cores by molding, at least a portion of the shell surrounds the cores such that the shell inner surface resides substantially conformally upon the outer surfaces of the cores. As used herein, the term “substantially conformally” means that the inner surface of the shell has peaks and valleys or indentations and protrusions corresponding substantially inversely to the peaks and valleys of the outer surface of the core. In certain such embodiments, the indentations and protrusions typically have a length, width, height or depth in one dimension of greater than 10 microns, say greater than 20 microns, and less than about 30,000 microns, preferably less than about 2000 microns.

The total weight of the shell is preferably about 20 percent to about 400 percent of the total weight of the cores. In embodiments wherein the shell is prepared by a solvent-free molding process, the total weight of the shell is typically from about 50 percent to about 400 percent, e.g. from about 75 percent to about 400 percent, or about 100 percent to about 200 percent of the total weight of the cores. In embodiments wherein the shell is prepared by a solvent-based molding process, the total weight of the shell is typically from about 20 percent to about 100 percent of the total weight of the cores.

The thickness of the shell is important to the release properties of the dosage form. Typical shell thicknesses that may be employed are about 50 to about 4000 microns. In certain preferred embodiments, the shell has a thickness of less than 800 microns. In embodiments wherein the shell portion is prepared by a solvent-free molding process, the shell portion typically has a thickness of about 500 to about 4000 microns, e.g. about 500 to about 2000 microns, say about 500 to about 800 microns, or about 800 to about 1200 microns. In embodiments wherein the shell portion is prepared by a solvent-based molding process, the shell portion typically has a thickness of less than about 800 microns, e.g. about 100 to about 600 microns, say about 150 to about 400 microns. In a particularly preferred embodiment the dosage form comprises first and second cores and first and second shell portions, and at least one of the shell portions has a thickness of less than about 800 microns, e.g. about 100 to about 600 microns, e.g. about 150 to about 400 microns.

In embodiments in which the shell is prepared by molding, the shell is substantially devoid of pores. Preferably, the fill or shell materials are typically substantially free of pores in the diameter range of 0.5 to 5.0 microns, i.e. has a pore volume in the pore diameter range of 0.5 to 5.0 microns of less than about 0.02 cc/g, preferably less than about 0.01 cc/g, more preferably less than about 0.005 cc/g. Typical compressed materials have pore volumes in this diameter range of more than about 0.02 cc/g.

Pore volume, pore diameter and density may be determined using a Quantachrome Instruments PoreMaster 60 mercury intrusion porosimeter and associated computer software program known as “Porowin.” The procedure is documented in the Quantachrome Instruments PoreMaster Operation Manual. The PoreMaster determines both pore volume and pore diameter of a solid or powder by forced intrusion of a non-wetting liquid (mercury), which involves evacuation of the sample in a sample cell (penetrometer), filling the cell with mercury to surround the sample with mercury, applying pressure to the sample cell by: (i) compressed air (up to 50 psi maximum); and (ii) a hydraulic (oil) pressure generator (up to 60000 psi maximum). Intruded volume is measured by a change in the capacitance as mercury moves from outside the sample into its pores under applied pressure. The corresponding pore size diameter (d) at which the intrusion takes place is calculated directly from the so-called “Washburn Equation”: d=−(4γ(cos θ)/P) where γ is the surface tension of liquid mercury, θ is the contact angle between mercury and the sample surface and P is the applied pressure.

In those embodiments in which solvent-free molding is employed, the flowable material may comprise a thermal-reversible carrier. Suitable thermal-reversible carriers for use in making a core, the shell or both by molding are thermoplastic materials typically having a melting point below about 110° C., more preferably between about 20 and about 100° C. Examples of suitable thermal-reversible carriers for solvent-free molding include thermoplastic polyalkylene glycols, thermoplastic polyalkylene oxides, low melting hydrophobic materials, thermoplastic polymers, thermoplastic starches, and the like. Preferred thermal-reversible carriers include polyethylene glycol and polyethylene oxide. Suitable thermoplastic polyalkylene glycols for use as thermal-reversible carriers include polyethylene glycol having molecular weight from about 100 to about 20,000, e.g. from about 100 to about 8,000 Daltons.

Suitable thermoplastic polyalkylene oxides include polyethylene oxide having a molecular weight from about 100,000 to about 900,000 Daltons. Suitable low-melting hydrophobic materials for use as thermal-reversible carriers include fats, fatty acid esters, phospholipids, and waxes which are solid at room temperature, fat-containing mixtures such as chocolate; and the like. Examples of suitable fats include hydrogenated vegetable oils such as for example cocoa butter, hydrogenated palm kernel oil, hydrogenated cottonseed oil, hydrogenated sunflower oil, and hydrogenated soybean oil; and free fatty acids and their salts. Examples of suitable fatty acid esters include sucrose fatty acid esters, mono, di, and triglycerides, glyceryl behenate, glyceryl palmitostearate, glyceryl monostearate, glyceryl tristearate, glyceryl trilaurylate, glyceryl myristate, GlycoWax-932, lauroyl macrogol-32 glycerides, and stearoyl macrogol-32 glycerides. Examples of suitable phospholipids include phosphotidyl choline, phosphotidyl serene, phosphotidyl enositol, and phosphotidic acid. Examples of suitable waxes that are solid at room temperature include carnauba wax, spermaceti wax, beeswax, candelilla wax, shellac wax, microcrystalline wax, and paraffin wax.

Suitable thermoplastic polymers for use as thermal-reversible carriers include thermoplastic water swellable cellulose derivatives, thermoplastic water insoluble polymers, thermoplastic vinyl polymers, thermoplastic starches, and thermoplastic resins, and combinations thereof. Suitable thermoplastic water swellable cellulose derivatives include hydroxypropylmethyl cellulose (HPMC), methyl cellulose (MC), carboxymethylcellulose (CMC), cross-linked hydroxypropylcellulose, hydroxypropyl cellulose (HPC), hydroxybutylcellulose (HBC), hydroxyethylcellulose (HEC), hydroxypropylethylcellulose, hydroxypropylbutylcellulose, hydroxypropylethylcellulose, and salts, derivatives, copolymers, and combinations thereof. Suitable thermoplastic water insoluble polymers include ethylcellulose, polyvinyl alcohols, polyvinyl acetate, polycaprolactones, cellulose acetate and its derivatives, acrylates, methacrylates, acrylic acid copolymers, and the like and derivatives, copolymers, and combinations thereof Suitable thermoplastic vinyl polymers include polyvinylacetate, polyvinyl alcohol, and polyvinyl pyrrolidone (PVP). Examples of suitable thermoplastic starches for use as thermal-reversible carriers are disclosed for example in U.S. Pat. No. 5,427,614. Examples of suitable thermoplastic resins for use as thermal-reversible carriers include dammars, mastic, rosin, shellac, sandarac, and glycerol ester of rosin. In one embodiment, the thermal-reversible carrier for making a core by molding is selected from polyalkylene glycols, polyalkylene oxides, and combinations thereof.

In embodiments in which the shell comprises an active ingredient intended to have immediate release from the dosage form, the shell is preferably prepared via solvent-free molding. In such embodiments a thermal-reversible carrier is employed in the flowable material to make the shell, said thermal-reversible carrier preferably selected from polyethylene glycol with weight average molecular weight from about 1450 to about 20000, polyethylene oxide with weight average molecular weight from about 100,000 to about 900,000, and the like.

In certain embodiments of the invention, the shell, or a shell portion may function as a diffusional membrane which contains pores through which fluids can enter the dosage form, contact and dissolve active ingredient in the core, which can then be released in a sustained, extended, prolonged or retarded manner. In these embodiments, the rate of release of active ingredient from an underlying core portion will depend upon the total pore area in the shell portion, the path length of the pores, and the solubility and diffusivity of the active ingredient (in addition to its rate of release from the core portion itself). In preferred embodiments in which a shell portion functions as a diffusional membrane, or is used for the wall of the osmotic chamber, the release of the active ingredient from the dosage form may be described as controlled, prolonged, sustained or extended. In these embodiments, the contribution to active ingredient dissolution from the shell may follow zero-order, first-order, or square root of time kinetics. In certain such embodiments, the shell portion preferably comprises a release modifying moldable excipient comprising a combination of a pore former and an insoluble edible material, for example a film forming water insoluble polymer. Alternately, in embodiments in which the shell portion is prepared by solvent-free molding, described below, the shell portion may comprise a thermal-reversible carrier that functions by dissolving and forming pores or channels through which the active ingredient may be liberated.

In certain other embodiments, the shell or a shell portion functions as an eroding matrix from which active ingredient dispersed in the shell is liberated by the dissolution of successive layers of the shell surface. In these embodiments, the rate of active ingredient release will depend on the dissolution rate of the matrix material in the shell or shell portion. Particularly useful matrix materials for providing surface erosion include those that first absorb liquid, then swell and/or gel prior to dissolving. In certain such embodiments, the shell or shell portion preferably comprises a release modifying moldable excipient comprising a swellable erodible hydrophilic material.

In certain other embodiments, the shell or a portion thereof functions as a barrier to prevent release therethrough of an active ingredient contained in an underlying core. In such embodiments, active ingredient is typically released from a portion of the core that is not covered by that portion of the shell, for example from a portion of the core in communication with one or more openings in the shell. Such embodiments advantageously allow for control of the surface area for release of the active ingredient. In certain embodiments for example, the surface area for release of active ingredient can be maintained substantially constant over time. In a particularly preferred embodiment, the release of at least one active ingredient follows substantially zero-order kinetics. In such embodiments, the shell preferably comprises a modified release composition comprising a water insoluble material, for example a water insoluble polymer.

In other embodiments, the shell, or a shell portion functions as a delayed release coating to delay release of one or more active ingredients contained in an underlying core or osmotic chamber. In these embodiments, the lag-time for onset of active ingredient release may be governed by erosion of the shell, diffusion of active ingredient through the shell, or a combination thereof. In certain such embodiments, the shell preferably comprises a release modifying moldable excipient comprising a swellable erodible hydrophilic material.

The following non-limiting example further illustrates the claimed invention.

### EXAMPLE

A dosage form according to the invention providing an immediate release of ibuprofen and an osmotic release of diphenhydramine is as follows.

Part A. Preparation of the 200 mg Immediate-Release (IR) Ibuprofen Core Formulation:

| Ingredients | Trade Name | Manufacturer | Mg/Tablet |
|---|---|---|---|
| Ibuprofen granules | | Albemarle Corp. | 200.0 |
| (115 microns) | | Orangeburg, SC | |
| Sodium starch | Explotab ® | Penwest | 12.0 |
| glycolate | | Pharmaceuticals | |
| | | Co. Patterson, NJ | |
| Colloidal silicon | Cab-O-Sil LM-5 ® | Cabot Corp. | 1.0 |
| dioxide | | Tuscola, IL | |
| Total | | | 213.0 |

Manufacturing Process:

Ibuprofen and sodium starch glycolate are delumped through a 30 mesh screen and said ingredients are mixed in a 2 qt. P-K blender for 5 minutes. Colloidal silicon dioxide is also delumped through a 30 mesh screen and is added to the aforementioned mixture for blending for another 5 minutes. Prescreened (through a 30 mesh screen) ibuprofen and sodium starch glycolate are mixed in a 2 qt. twin-shell blender for 5 minutes.

The final blend (from Step 1) is fed into the die of a rotary tablet press and is compressed into a tablet core under 2000 lb/in² of operating pressure. The weight of compressed tablet is 213.0 mg, which contains 200.0 mg of ibuprofen.

Part B: Preparation of the Osmotic Core

1. A core having the formula set forth in Table B below was prepared as follows:

TABLE B
2. Formula of Core Portion:

| Ingredient | Trade Name | Manufacturer | Weight % |
|---|---|---|---|
| Polyethylene Oxide | Polyox ® | The Dow Chemical | 75 |
| (MW = 300,000) | WSR 750 NF | Company, Midland, | |
| | | MI | |
| Diphenhydramine | | Kongo | 15 |
| HCl, USP | | | |
| Hydroxypropyl | Methocel E5 | Dow Chemical | 8.5 |
| Methylcellulose | | Company, Midland, | |
| | | MI | |
| Magnesium | — | Mallinckrodt, Inc | 1.5 |
| stearate | | | |
| D&C Yellow #10 | D&C Yellow | Colorcon Inc, | Trace |
| | #10 HT | Westpoint, Pa | Amount |
| Isopropyl Alcohol | — | EM Science | |
| (dried as solvent) | | | |

3. The diphenhydramine HCl, hydroxypropyl methylcellulose, and PEO (MW=300,000) are first mixed in a plastic bag for 1-2 minutes. After this powder mixture is added into a 5 qt. bowl of a Hobart planetary mixer, the alcohol is added thereto with mixing at about 60 rpm. After mixing the ingredients for about 10 minutes, the resulting granulation is removed from the bowl and dried at room temperature for 12 to 16 hours to remove all residual solvent. The granulation is then screened through a #20 mesh screen and put into a plastic bag. Magnesium stearate was added to the dry granules, followed by mixing for 3 minutes.

4. The resulting diphenhydramine HCl granulation (440 mg) is then fed into a rotary tablet press equipped with round, concave compression punch and die units having a 0.4375″ diameter. The granulation is pressed into solid tablets using 2000 lb/sq. in. of compression force.

5. Part C: Preparation of Shell Portion by Solvent based molding The shell according to following tablet was prepared as follows.

| Ingredient | Trade Name | Manufacturer | Weight %* |
|---|---|---|---|
| Water | — | — | 17.17 |
| Acetone | B&J Brand R High | Honeywell | 40.08 |
| | Purity Solvent | International Inc., | |
| | | Muskegon, MI | |
| Cellulose | Cellulose Acetate, | Eastman Chemical | 22.90 |
| Acetate | NF | Company, Kingsport, | |
| | | TN | |
| Carrageenan | Gelcarin GP-812, | FMC Corporation, | 0.76 |
| | NF | Pharmaceutical | |
| | | Division, Newark, | |
| | | DE | |
| Triacetin | Triacetin, Food | Eastman Chemical | 15.27 |
| | Grade | Company, Kingsport, | |
| | | TN | |
| Polyethylene | Polyethylene | The Dow Chemical | 3.82 |
| Glycol 400 | Glycol 400 NF, | Company, Midland, | |
| | FCC Grade | MI | |

*weight percentage of active ingredient based upon total wet weight of the polymeric composition

6. The cellulose acetate is added to a beaker containing acetone, triacetin, polyethylene glycol, and water and mixed using a mixer until all powder is dissolved. The mixture is then heated in the 55 ° C. water bath to obtain a viscous solution. The carrageenan is then added to the hot solution, and the resulting mixture was heated and stirred until a homogeneous texture was obtained.

Part D: Preparation of Fill Material: for Immediate Release

The fill material is prepared for application to the first core portion prepared in Part A. The fill material comprises red gelatin for immediate release, and is made of the following ingredients: purified water, Opatint Red DD-1761, and 275 Bloom Pork Skin Gelatin added together as a mix of dry gelatin granules. A gelatin slurry is formed from these ingredients and heated to 55° C. to melt and dissolve the gelatin. The gelatin is solution is held at 55° C. for approximately 3 hours (holding times at this temperature can generally range between about 2 and about 16 hours). The solution is then mixed until uniform (about 5 to 15 minutes). The gelatin solution is maintained at 55° C. with continuous mixing during its use in the first thermal cycling molding module.

Laboratory Manufacturing Process for Application of the Shell and Fill Material:

A laboratory scale thermal cycle molding unit having an overall caplet shape of dimensions of 0.700″×0.350″×0.06″, is used to apply the shell portion to the cores. The molding unit comprises a single mold assembly made from an upper mold assembly portion comprising an upper mold cavity, and a lower mold assembly portion comprising a lower mold cavity. The lower mold assembly portion is first cooled to 5° C. The shell material of Part C is introduced into the lower mold cavity. Two separate cores prepared, as described in aforementioned Parts A and B, are immediately inserted into two stations within the cavity. The in-process dosage form is held in the chilled mold for 20 seconds to allow the shell material to harden. The stations separate the two cores within the lower mold cavity by 1 mm.

A blank upper mold assembly portion is mated with the lower mold assembly portion. The upper mold cavity comprises a small rod (0.1 mm in diameter×1 mm in length) attached to its inner surface that contacts one station for core of Part A (200 mg ibuprofen tablet) to allow a portion of the dosage form to remain uncoated, and a second small rod that contacts one station for the core of Part B (Osmotic diphenhydramine tablet). The shell material of Part C is introduced into the upper mold cavity. The lower mold assembly portion, which has been maintained at 5° C., is mated with the upper mold assembly portion in such a way that the cores of Part A (200 mg ibuprofen tablet) and Part B (Osmotic diphenhydramine tablet) is mated with the first core station of the upper mold assembly. The shell material of Part C is introduced into the lower mold cavity and held at 5° C. for 30 seconds to harden.

The fill material portion is injected into the upper mold portion and covers the portion of the core from Part A that was not previously covered by the shell to allow for immediate release. A small orifice remains over the core from Part B to allow for osmotic release. The upper mold is held at 5° C. for 60 seconds to allow the first and second fill material portions to harden. The lower mold assembly portion is then removed and the finished dosage form, a molded caplet coated with a shell material and two fill materials, is ejected from the upper mold cavity. The weight gain from the shell material (i.e. the difference in weight between the finished dosage form and the core) is recorded.

Manufacturing Process for Application of the Shell and Fill Material:

Dosage forms of the invention are made in a continuous process using an apparatus comprising two thermal cycle molding modules linked in series via a transfer device as described at pages 14-16 of copending U.S. application Ser. No. 09/966,939, the disclosure of which is incorporated herein by reference. The dosage forms comprise two cores coated with a shell and a first and second fill portion.

The thermal cycle molding modules have the general configuration shown in FIG. 3 and pages 27-51 of copending U.S. application Ser. No. 09/966,497, which depicts a thermal cycle molding module 200 comprising a rotor 202 around which a plurality of mold units 204 are disposed. The thermal cycle molding modules include reservoirs 206 (see FIG. 4) for holding the shell material, the first fill material, and the second fill material. In addition, each thermal cycle molding module is provided with a temperature control system for rapidly heating and cooling the mold units. FIGS. 55 and 56 of pending U.S. application Ser. No. 09/966,497 depict the temperature control system 600.

The transfer device has the structure shown as 300 in FIG. 3 and described on pages 51-57 of copending U.S. application Ser. No. 09/966,414, the disclosure of which is incorporated by reference. It comprises a plurality of transfer units 304 attached in cantilever fashion to a belt 312 as shown in FIGS. 68 and 69. The transfer device rotates and operates in sync with the thermal cycle molding modules to which it is coupled. Transfer units 304 comprise retainers 330 for holding the cores as they travel around the transfer device.

The transfer device transfers the cores aforementioned in Part A and Part B to the second molding module, which applies the shell to the cores. The second thermal cycle molding module is of the type shown in FIG. 28A of copending U.S. application Ser. No. 09/966,497. The mold units 204 of the second thermal cycle molding module comprise upper mold assemblies 214, rotatable center mold assemblies 212 and lower mold assemblies 210 as shown in FIG. 28C. Cores are continuously transferred to the mold assemblies, which then close over the cores.

At the beginning of the molding cycle (rotor at the 0 degree position) the mold assemblies are in the open position. Center mold assembly 212 as shown in copending U.S. application Ser. No. 09/966,497 as incorporated herein by reference, has received the compressed cores, for example from a compression module according to the invention transferred via a transfer device also according to the invention. As the rotor continues to revolve, the upper mold assembly 214 closes against center mold assembly 212. Next, flowable material is injected into the mold cavity created by union of the mold assemblies to apply a shell from Part C to the first half of the dosage form. The flowable material is cooled in the mold cavity. The mold assemblies open with the partially coated dosage forms remaining in the upper mold assembly 214. Upon further revolution of the rotor, the center mold assembly rotates 180 degrees. As the rotor moves past 180 degrees the mold assemblies again close and the uncoated portion of the compressed dosage form is covered with flowable material thus forming a shell having an opening aligned with the fill material, in a mold assembly that contains one or more protrusions, which prevent a portion of the cores from being covered. The protrusions also contain nozzles for injecting the fill materials. The protrusions retract following the application of the second portion of the shell from Part C, and the flowable fill material from Part D, heated to a flowable state in reservoirs 206, are injected into the uncoated portions over the core of Part A, forming the fill portions 16 as shown in FIG. 2. A small orifice remains over the core from Part B to allow for osmotic release. A molding cycle is completed with setting or hardening of the shell and fill materials on the second half of the compressed dosage form. The mold assemblies again open and the coated compressed dosage form is ejected from the molding module.

Although this invention has been illustrated by reference to specific embodiments, it will be apparent to those skilled in the art that various changes and modifications may be made which clearly fall within the scope of this invention.

We claim:

1. A dosage form comprising a first immediate release core containing at least one pharmaceutically active ingredient, an osmotic core containing at least one pharmaceutically active ingredient that is the same or different from the pharmaceutically active ingredient provided in the first core, and a unitary shell that conforms and coats at least a substantial portion of both the first immediate release core and the osmotic core, wherein the shell material is substantially impermeable to the pharmaceutically active ingredient in the osmotic core.

2. A dosage form according to claim 1 wherein at least one passageway is provided through the shell to the immediate release core with sufficient size for immediate release of the active contained therein and at least one passageway is provided through the unitary shell to the osmotic chamber.

3. A dosage form according to claim 2, wherein the passageways are provided with a fill material that is compositionally different from the shell.

4. A dosage form according to claim 1, wherein the first immediate release core is a multi-layer tablet.

5. A dosage form according to claim 1, wherein the pore volume of the shell as applied is less than 0.2 cc/g for pores having a diameter between about 0.5 to about 5 microns.

6. A dosage form comprising a first immediate release core containing at least one pharmaceutically active ingredient, an osmotic core containing at least one pharmaceutically active ingredient that is the same or different from the pharmaceutically active ingredient provided in the first immediate release core, and a shell having distinct, compositionally different portions and that conforms and coats at least a substantial portion of both the first immediate release core and the osmotic core, wherein the shell has a major portion consisting essentially of material that is substantially impermeable to the pharmaceutically active ingredient in the osmotic chamber and a second portion of the shell in contact with the immediate release core that consists essentially of immediate release material.

6. A dosage form according to claim 6 wherein at least one passageway is provided through the shell to the osmotic chamber.

7. A dosage form according to claim 6, wherein the first immediate release core comprises a compressed solid tablet.

8. A dosage form according to claim 6, wherein the first immediate release core is a multi-layer tablet.

9. A dosage form according to claim 6, wherein all portions of the shell are substantially free of pores having a diameter of 0.5 to 5.0 microns.

10. A dosage form according to claim 6, wherein the pore volume of all portions of the shell as applied is less than 0.2 cc/g for pores having a diameter between about 0.5 to about 5 microns.

11. A dosage form comprising a first immediate release core containing at least one pharmaceutically active ingredient, an osmotic core containing at least one pharmaceutically active ingredient that is the same or different from the pharmaceutically active ingredient provided in the first immediate release core, and a unitary shell that conforms and coats at least a substantial portion of the first immediate release core and the osmotic core, wherein a portion of the shell provided over the immediate release core is sufficiently thin that it ruptures upon swelling of the immediate release core to release the active contained therein.

12. A dosage form according to claim 11 wherein at least one passageway is provided through the shell to the osmotic chamber.

13. A dosage form according to claim 1 1, wherein the first immediate release core comprises a compressed solid tablet.

14. A dosage form according to claim 11, wherein the shell is substantially free of pores having a diameter of 0.5 to 5.0 microns.

15. A dosage form according to claim 1 1, wherein the pore volume of the shell as applied is less than 0.2 cc/g for pores having a diameter between about 0.5 to about 5 microns.

16. A dosage form comprising a first immediate release core containing a pharmaceutically active ingredient, an osmotic chamber containing one pharmaceutically active ingredient that is the same or different from the pharmaceutically active ingredient provided in the first core, and a shell that consists essentially of an immediate release material and coats at least a substantial portion of the first immediate release core and the osmotic chamber, wherein the osmotic chamber comprises a barrier layer that is substantially impermeable to the active ingredient contained therein.

17. A dosage form according to claim 16 wherein a passageway is provided through the barrier layer.

18. A dosage form according to claim 16, wherein the first immediate release core comprises a compressed solid tablet.

19. A dosage form according to claim 16, wherein the shell as applied is substantially free of pores having a diameter of 0.5 to 5.0 microns.

20. A dosage form according to claim 16, wherein the pore volume of shell as applied is less than 0.2 cc/g for pores having a diameter between about 0.5 to about 5 microns.

21. A dosage form comprising a first core containing a pharmaceutically active ingredient, an osmotic chamber containing one pharmaceutically active ingredient that is the same or different from the pharmaceutically active ingredient provided in the first core, and a shell having distinct, compositionally different portions that coat a substantial portion of the first core and osmotic chamber, wherein the first shell portion that is in contact with the first core provides an immediate release of the pharmaceutically active ingredient in the first core and the second shell portion in contact with the osmotic chamber produces a modified release profile.

22. A dosage form according to claim 21, wherein the first immediate release core comprises a compressed solid tablet.

23. A dosage form according to claim 21, wherein the first immediate release core is a multi-layer tablet.

24. A dosage form according to claim 21, wherein all portions of the shell are substantially free of pores having a diameter of 0.5 to 5.0 microns.

25. A dosage form according to claim 21, wherein the pore volume of all portions of the shell as applied are less than 0.2 cc/g for pores having a diameter between about 0.5 to about 5 microns.

26. A method for preparing a dosage form comprising:
a) providing a first immediate release core containing at least one pharmaceutically active ingredient and an osmotic core or an osmotic chamber containing at least one pharmaceutically active ingredient that is the same or different from the pharmaceutically active ingredient provided in the first core to shell-forming module,
b) providing a shell that conforms and coats at least a substantial portion of both the first immediate release core and the osmotic core or osmotic chamber.

27. A method for preparing a dosage form comprising:
a) providing a first immediate release core containing at least one pharmaceutically active ingredient and an osmotic core or an osmotic chamber containing at least one pharmaceutically active ingredient that is the same or different from the pharmaceutically active ingredient provided in the first core to a shell-forming module,
b) providing a shell having distinct portions and that conforms and coats at least a substantial portion of both the first immediate release core and the osmotic core or osmotic chamber.
